(19) United States
(12) Patent Application Publication
(10) Pub. No.: US 2021/0269414 A1
(43) Pub. Date: Sep. 2, 2021

(54) PROCESS FOR PREPARING TRICYCLIC COMPOUNDS

(71) Applicant: Bayer Aktiengesellschaft, Leverkusen (DE)

(72) Inventors: Andreas REMBIAK, Bad Soden (DE); Kira HEIL, Dormagen (DE); Andreas Karl STEIB, Wuppertal (DE); Mark James FORD, Niedernhausen (DE)

(73) Assignee: Bayer Aktiengesellschaft, Leverkusen (DE)

(21) Appl. No.: 17/254,729

(22) PCT Filed: Jun. 17, 2019
(86) PCT No.: PCT/EP2019/065854

§ 371 Date: Dec. 21, 2020

(30) Foreign Application Priority Data
Jun. 22, 2018 (EP) 18179216.9

(51) Int. Cl. C07D 401/04

(52) U.S. Cl. CPC C07D 401/04

(57) ABSTRACT

The present invention relates to a process for preparing compounds of the formula (I)

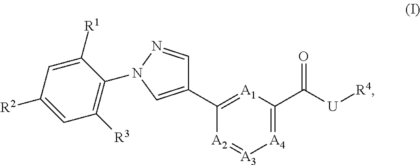

starting from compounds of the formula (II)

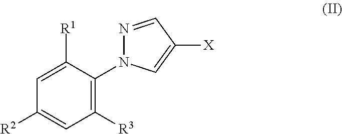

wherein X, R¹, R², R³, R⁴, U, A₁, A₂, A₃ and A₄ are defined as described above.

The invention relates to compounds of the formulae (III) and (IV)

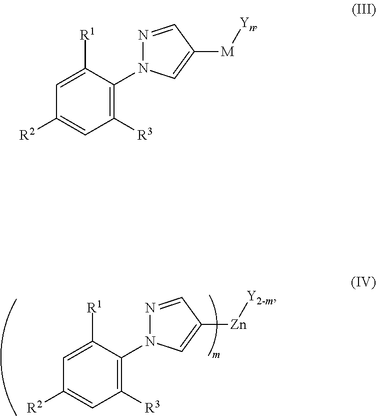

wherein all variables are defined as above.

The present invention relates to a process for preparing tricyclic compounds of the formula (I)

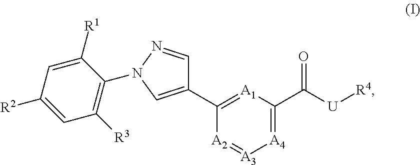

starting from compounds of the formula (II)

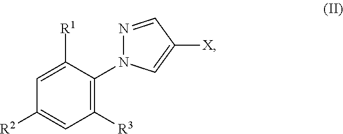

in which X, R¹, R², R³, R⁴, U, A₁, A₂, A₃ and A₄ are defined as described below.

The preparation of compounds of formula (I) is known, for example, from WO2018/104214, WO2015/067646, WO2015/067647 or WO2016/174052. The preparation takes place in this case by means of palladium-catalysed Suzuki-coupling using pyrazoleboronic acid derivatives or the corresponding arylboronic acid derivatives. Disadvantages in this process are the use of costly, high catalyst loadings of from 5 to 10 mol % of palladium and also the technically complex preparation and necessary isolation of the boronic acid derivatives, some of which are not very stable.

Because of the importance of tricyclic compounds of the general formula (I) as novel agrochemical active ingredients or precursors for these, the object of the present invention is that of providing a process for preparing compounds of the general formula (I) which can be used industrially and cost-effectively and avoids the above-described disadvantages, especially the high catalyst loading and the difficult isolation of the boronic acid derivatives. It is also desirable to obtain the specific N-arylpyrazole derivatives with high yield and high purity, such that the target compound preferably does not have to be subjected to any further potentially complex purification.

This object was achieved according to the invention by a process for preparing compounds of the formula (I)

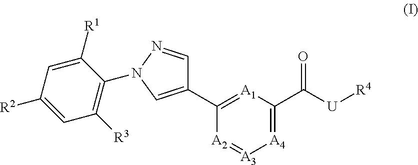

in which

R¹ is hydrogen, cyano, halogen, C₁-C₄-alkyl optionally substituted by halogen or CN, or C₁-C₄-alkoxy optionally substituted by halogen,

R² is halogen, trifluoromethylsulfonyl, trifluoromethylsulfinyl, trifluoromethylsulfanyl, C₁-C₄-alkyl optionally substituted by halogen, or C₁-C₄-alkoxy optionally substituted by halogen and

R³ is hydrogen, cyano, halogen, C₁-C₄-alkyl optionally substituted by halogen or CN, or C₁-C₄-alkoxy optionally substituted by halogen,

R⁴ is hydrogen, C₁-C₅-alkyl optionally substituted by halogen or CN, or C₃-C₆-cycloalkyl optionally substituted by halogen or CN,

U is oxygen or an N(R⁵) group, where

R⁵ is hydrogen, C₁-C₆-alkyl optionally substituted by halogen or CN, or C₃-C₆-cycloalkyl optionally substituted by halogen or CN,

A₁ is C—R₆,

A₂ is C—R₇,

A₃ is C—R₈ or N,

A₄ is C—R₉,

R₆, R₇, R₈ and R₉ each independently of one another are hydrogen, C₁-C₄-alkyl optionally substituted by halogen or CN, or halogen,

starting from compounds of the formula (II)

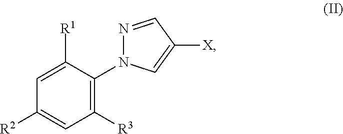

where R¹, R² and R³ are as defined above and X is iodine or bromine, comprising the steps of
(1): reacting the compounds of the formula (II) with a magnesium- or lithium-based metallation reagent to give compounds of the formula (III)

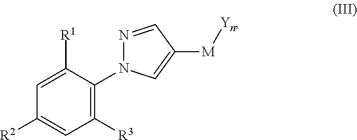

where R¹, R², R³ are as defined above, Y is chlorine, bromine or iodine, n is 0 or 1 and M is magnesium (when n=1) or lithium (when n=0),
(2): reacting the compounds of the formula (III) with an inorganic zinc compound to give compounds of the formula (IV)

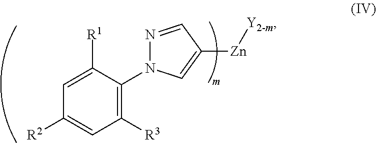

where R¹, R², R³ and Y are as defined above and m is 1 or 2, and
(3): reacting compounds of the formula (IV) with compounds of the formula (V)

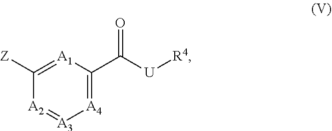

where A₁, A₂, A₃, A₄, R⁴, U and R⁵ are as defined above and Z is bromine or iodine, in the presence of at least one Pd(0) or Pd(II) compound and at least one monodentate or bidentate ligand, to give compounds of the formula (I).

The process according to the invention has the advantage over the previously described process that, firstly, isolation of the reactive species and thus an additional reaction step can be dispensed with, the catalyst loading and therefore the impact on the environment and on costs can be considerably reduced, and the target compounds of the general form (I) can be obtained without a complex purification step in good yields and high purities.

The preferred embodiments described below refer, if applicable, to all formulae described herein.

In one preferred configuration of the invention, R₆, R₇, R₈ and R₉ in each case independently of one another are hydrogen, methyl or halogen.

In one additionally preferred configuration of the invention, at most two, particularly preferably at most one, of the radicals R₆, R₇, R₈ and R₉ are not hydrogen. Particularly preferably, R₆, R₇ and R₈ are hydrogen and R₉ is C₁-C₄-alkyl optionally substituted by halogen or CN, or halogen.

In one very particularly preferred embodiment, R₉ is halogen, especially Cl, F, I or Br, and specifically is Cl.

In one further preferred embodiment, A₃ is N.

In one further preferred embodiment,

A₁ is C—H,

A₂ is C—H,

A₃ is C—H or N and

A₄ is C—R₉,

where R₉ is as defined above.

In one further particularly preferred embodiment,

A₁ is C—H,

A₂ is C—H,

A₃ is N and

A₄ is C-halogen, preferably C—Cl, C—F, C—I, C—Br, more preferably C—Cl.

If U is O, the following preferred configurations for R⁴ apply:

In one particularly preferred configuration, R₄ is hydrogen or C₁-C₆-alkyl, very particularly preferably methyl or ethyl.

If U is N(R⁵), the following preferred configurations for R⁴ and R⁵ apply:

In one preferred embodiment of the invention, at most one of the radicals R⁴ or R⁵ is hydrogen.

In one further preferred configuration, R₄ is C₃-C₆-cycloalkyl optionally substituted by Cl, Br, I, F or CN, very particularly preferably cyclopropyl or 1-CN-cyclopropyl.

In one particularly preferred embodiment,

R₄ is C₃-C₆-cycloalkyl optionally substituted by Cl, Br, I, F or CN and

R₅ is hydrogen or C₁-C₄-alkyl optionally substituted by Cl, Br, I, F or CN.

Very particularly preferably,

R₄ is cyclopropyl or 1-CN-cyclopropyl and

R₅ is hydrogen or C₁-C₄-alkyl, especially methyl or ethyl.

In one preferred configuration of the invention, U is an N(R⁵) group.

In one preferred embodiment of the invention,
R² is halogen-substituted C₁-C₄-alkyl or halogen-substituted C₁-C₄-alkoxy, such as for example difluoromethyl, trichloromethyl, chlorodifluoromethyl, dichlorofluoromethyl, trifluoromethyl, 1-fluoroethyl, 2-fluoroethyl, 2,2-difluoroethyl, 2,2,2-trifluoroethyl, 1,2,2,2-tetrafluoroethyl, 1-chloro-1,2,2,2-tetrafluoroethyl, 2,2,2-trichloroethyl, 2-chloro-2,2-difluoroethyl, 1,1-difluoroethyl, pentafluoroethyl, pentafluoro-tert-butyl, heptafluoro-n-propyl, heptafluoroisopropyl, nonafluoro-n-butyl, nonafluoro-sec-butyl, fluoromethoxy, difluoromethoxy, chlorodifluoromethoxy, dichlorofluoromethoxy, trifluoromethoxy, 2,2,2-trifluoroethoxy, 2-chloro-2,2-difluoroethoxy or pentafluoroethoxy.

Particularly preferably,
R² is fluorine-substituted C₁-C₄-alkyl or fluorine-substituted C₁-C₄-alkoxy.

Very particularly preferably,
R² is perfluoro-C₁-C₃-alkyl (CF₃, C₂F₅ or C₃F₇ (n- or isopropyl)) or perfluoro-C₁-C₃-alkoxy (OCF₃, OC₂F₅ or OC₃F₇ (n- or isopropoxy)).

Especially preferably,
R² is perfluoro-C₁-C₃-alkyl, such as trifluoromethyl, pentafluoroethyl, heptafluoroisopropyl or heptafluoro-n-propyl, especially heptafluoroisopropyl.

In one further preferred embodiment, R¹ and R³ in each case independently of one another are a substituent selected from hydrogen, Cl, Br, F, C₁-C₃-alkyl, halogen-substituted C₁-C₃-alkyl, C₁-C₃-alkoxy or halogen-substituted C₁-C₃-alkoxy.

In one further preferred embodiment, R¹ and R³ are the substituents described herein, but R¹ and R³ are not simultaneously hydrogen in any compound. In other words, when R¹ in a compound is hydrogen, R³ is one of the other substituents described herein, and vice versa.

In one particularly preferred embodiment, R¹ and R³ in each case independently of one another are Cl, Br, C₁-C₃-alkyl, or fluorine-substituted C₁-C₃-alkyl, C₁-C₃-alkoxy or fluorine-substituted C₁-C₃-alkoxy, especially Cl, Br, methyl, trifluoromethyl, trifluoromethoxy or difluoromethoxy.

In one very particularly preferred embodiment, R¹ and R³ independently of one another are Cl, Br or F, especially Cl or Br. In one particularly advantageous configuration of the invention, R¹ and R³ are the same halogen, especially chlorine.

In one preferred configuration of the invention, at least one of the radicals R¹, R², R³ is halogen-substituted C₁-C₄-alkyl or halogen-substituted C₁-C₄-alkoxy, particularly preferably fluorine-substituted C₁-C₃-alkyl or fluorine-substituted C₁-C₃-alkoxy.

In one further particularly advantageous configuration of the invention,
R¹ is halogen or C₁-C₃-alkyl, especially Br, Cl or methyl,
R² is fluorine-substituted C₁-C₄-alkyl or fluorine-substituted C₁-C₄-alkoxy, especially heptafluoroisopropyl, and
R³ is halogen, C₁-C₃-alkyl or fluorine-substituted C₁-C₃-alkyl, C₁-C₃-alkoxy or fluorine-substituted C₁-C₃-alkoxy, especially Cl, methyl, trifluoromethyl, trifluoromethoxy or difluoromethoxy.

In one additionally preferred configuration of the invention,
R¹ is halogen or C₁-C₃-alkyl, especially Br, Cl or methyl,
R² is fluorine-substituted C₁-C₄-alkyl or fluorine-substituted C₁-C₄-alkoxy, especially heptafluoroisopropyl,
R³ is halogen, C₁-C₃-alkyl or fluorine-substituted C₁-C₃-alkyl, C₁-C₃-alkoxy or fluorine-substituted C₁-C₃-alkoxy, especially Cl, methyl, trifluoromethyl, trifluoromethoxy or difluoromethoxy,
R⁴ is hydrogen, C₁-C₆-alkyl or C₃-C₆-cycloalkyl optionally substituted by Cl, Br, I, F or CN, especially methyl, ethyl, cyclopropyl and 1-CN-cyclopropyl,
U is oxygen or an N(R⁵) group, where
R⁵ is hydrogen or C₁-C₄-alkyl, especially methyl or ethyl,
A₁ is C—H,
A₂ is C—H,
A₃ is C—H or N, especially N, and
A₄ is C—R₉, where
R₉ is halogen, especially Cl.

In one preferred configuration of the invention, X is iodine.

Compounds of the formula (II) are obtainable by halogenating the corresponding pyrazole derivatives. The preparation has already been described in WO2018/104214, WO2015/067646, WO2015/067647 or WO2016/174052.

Preferred halopyrazoles of the formula (II) are
4-bromo-1-[4-(1,1,1,2,3,3,3-heptafluoropropan-2-yl)-2,6-dimethylphenyl]-1H-pyrazole
4-bromo-1-[2,6-dichloro-4-(1,1,1,2,3,3,3-heptafluoropropan-2-yl)phenyl]-1H-pyrazole
4-bromo-1-[2-chloro-4-(1,1,1,2,3,3,3-heptafluoropropan-2-yl)-6-(trifluoromethyl)phenyl]-1H-pyrazole
4-bromo-1-[2-chloro-4-(1,1,1,2,3,3,3-heptafluoropropan-2-yl)-6-(trifluoromethoxy)phenyl]-1H-pyrazole
4-bromo-1-[2-chloro-6-(difluoromethoxy)-4-(1,1,1,2,3,3,3-heptafluoropropan-2-yl)phenyl]-1H-pyrazole
4-bromo-1-[4-(1,1,1,2,3,3,3-heptafluoropropan-2-yl)-2-methyl-6-(trifluoromethyl)phenyl]-1H-pyrazole
4-bromo-1-[2-bromo-4-(1,1,1,2,3,3,3-heptafluoropropan-2-yl)-6-(trifluoromethyl)phenyl]-1H-pyrazole
4-bromo-1-[2-bromo-4-(1,1,1,2,3,3,3-heptafluoropropan-2-yl)-6-(trifluoromethoxy)phenyl]-1H-pyrazole
1-[4-(1,1,1,2,3,3,3-heptafluoropropan-2-yl)-2,6-dimethylphenyl]-4-iodo-1H-pyrazole
1-[2,6-dichloro-4-(1,1,1,2,3,3,3-heptafluoropropan-2-yl)phenyl]-4-iodo-1H-pyrazole
1-[2-chloro-4-(1,1,1,2,3,3,3-heptafluoropropan-2-yl)-6-(trifluoromethyl)phenyl]-4-iodo-1H-pyrazole
1-[2-chloro-4-(1,1,1,2,3,3,3-heptafluoropropan-2-yl)-6-(trifluoromethoxy)phenyl]-4-iodo-1H-pyrazole
1-[2-chloro-6-(difluoromethoxy)-4-(1,1,1,2,3,3,3-heptafluoropropan-2-yl)phenyl]-4-iodo-1H-pyrazole
1-[4-(1,1,1,2,3,3,3-heptafluoropropan-2-yl)-2-methyl-6-(trifluoromethyl)phenyl]-4-iodo-1H-pyrazole
1-[2-bromo-4-(1,1,1,2,3,3,3-heptafluoropropan-2-yl)-6-(trifluoromethyl)phenyl]-4-iodo-1H-pyrazole
1-[2-bromo-4-(1,1,1,2,3,3,3-heptafluoropropan-2-yl)-6-(trifluoromethoxy)phenyl]-4-iodo-1H-pyrazole Particular preference is given here to the following compounds:
4-bromo-1-[2,6-dichloro-4-(1,1,1,2,3,3,3-heptafluoropropan-2-yl)phenyl]-1H-pyrazole
4-bromo-1-[2-chloro-4-(1,1,1,2,3,3,3-heptafluoropropan-2-yl)-6-(trifluoromethyl)phenyl]-1H-pyrazole
4-bromo-1-[2-chloro-4-(1,1,1,2,3,3,3-heptafluoropropan-2-yl)-6-(trifluoromethoxy)phenyl]-1H-pyrazole
4-bromo-1-[2-chloro-6-(difluoromethoxy)-4-(1,1,1,2,3,3,3-heptafluoropropan-2-yl)phenyl]-1H-pyrazole
4-bromo-1-[4-(1,1,1,2,3,3,3-heptafluoropropan-2-yl)-2-methyl-6-(trifluoromethyl)phenyl]-1H-pyrazole
4-bromo-1-[2-bromo-4-(1,1,1,2,3,3,3-heptafluoropropan-2-yl)-6-(trifluoromethyl)phenyl]-1H-pyrazole
4-bromo-1-[2-bromo-4-(1,1,1,2,3,3,3-heptafluoropropan-2-yl)-6-(trifluoromethoxy)phenyl]-1H-pyrazole
1-[2,6-dichloro-4-(1,1,1,2,3,3,3-heptafluoropropan-2-yl)phenyl]-4-iodo-1H-pyrazole
1-[2-chloro-4-(1,1,1,2,3,3,3-heptafluoropropan-2-yl)-6-(trifluoromethyl)phenyl]-4-iodo-1H-pyrazole
1-[2-chloro-4-(1,1,1,2,3,3,3-heptafluoropropan-2-yl)-6-(trifluoromethoxy)phenyl]-4-iodo-1H-pyrazole
1-[2-chloro-6-(difluoromethoxy)-4-(1,1,1,2,3,3,3-heptafluoropropan-2-yl)phenyl]-4-iodo-1H-pyrazole
1-[4-(1,1,1,2,3,3,3-heptafluoropropan-2-yl)-2-methyl-6-(trifluoromethyl)phenyl]-4-iodo-1H-pyrazole
1-[2-bromo-4-(1,1,1,2,3,3,3-heptafluoropropan-2-yl)-6-(trifluoromethyl)phenyl]-4-iodo-1H-pyrazole
1-[2-bromo-4-(1,1,1,2,3,3,3-heptafluoropropan-2-yl)-6-(trifluoromethoxy)phenyl]-4-iodo-1H-pyrazole

Compounds of the formula (V) can be obtained by esterification or amidation of the corresponding halogenated aryl- or N-heteroarylcarboxylic acid derivatives in analogy to the methods generally known to those skilled in the art, and their preparation has been described, for example, in WO2018/104214, WO2014/186398 or WO2013/042035.

Preferred compounds of the formula (V) are:
methyl 2-chloro-5-iodopyridine-3-carboxylate
methyl 5-bromo-2-chloropyridine-3-carboxylate
ethyl 2-chloro-5-iodopyridine-3-carboxylate
ethyl 5-bromo-2-chloropyridine-3-carboxylate
n-propyl 2-chloro-5-iodopyridine-3-carboxylate
n-propyl 5-bromo-2-chloropyridine-3-carboxylate
isopropyl 2-chloro-5-iodopyridine-3-carboxylate
isopropyl 5-bromo-2-chloropyridine-3-carboxylate
tert-butyl 2-chloro-5-iodopyridine-3-carboxylate
tert-butyl 5-bromo-2-chloropyridine-3-carboxylate
5-bromo-2-chloro-N,N-dimethylpyridine-3-carboxamide
2-chloro-5-iodo-N,N-dimethylpyridine-3-carboxamide
2-chloro-N-cyclopropyl-5-iodo-N-methylpyridine-3-carboxamide
5-bromo-2-chloro-N-cyclopropyl-N-methylpyridine-3-carboxamide
2-chloro-5-iodo-N-isopropyl-N-methylpyridine-3-carboxamide
5-bromo-2-chloro-N-isopropyl-N-methylpyridine-3-carboxamide
Particular preference is given here to:
methyl 2-chloro-5-iodopyridine-3-carboxylate
methyl 5-bromo-2-chloropyridine-3-carboxylate
ethyl 2-chloro-5-iodopyridine-3-carboxylate
ethyl 5-bromo-2-chloropyridine-3-carboxylate
n-propyl 2-chloro-5-iodopyridine-3-carboxylate
n-propyl 5-bromo-2-chloropyridine-3-carboxylate
2-chloro-N-cyclopropyl-5-iodo-N-methylpyridine-3-carboxamide
5-bromo-2-chloro-N-cyclopropyl-N-methylpyridine-3-carboxamide

Preference is given to preparing the following compounds of the formula (I) by means of the process described herein:

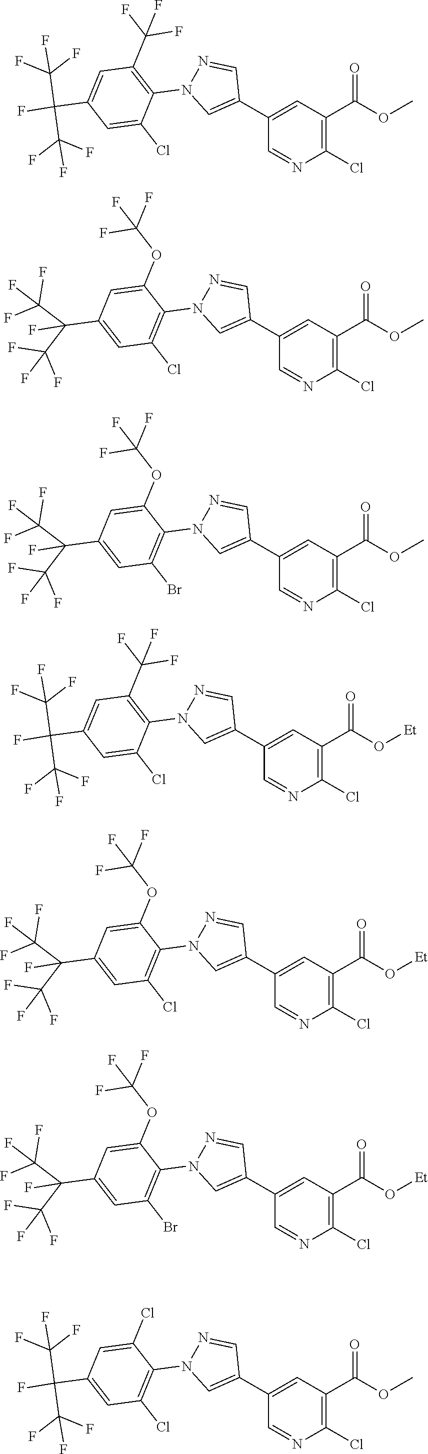

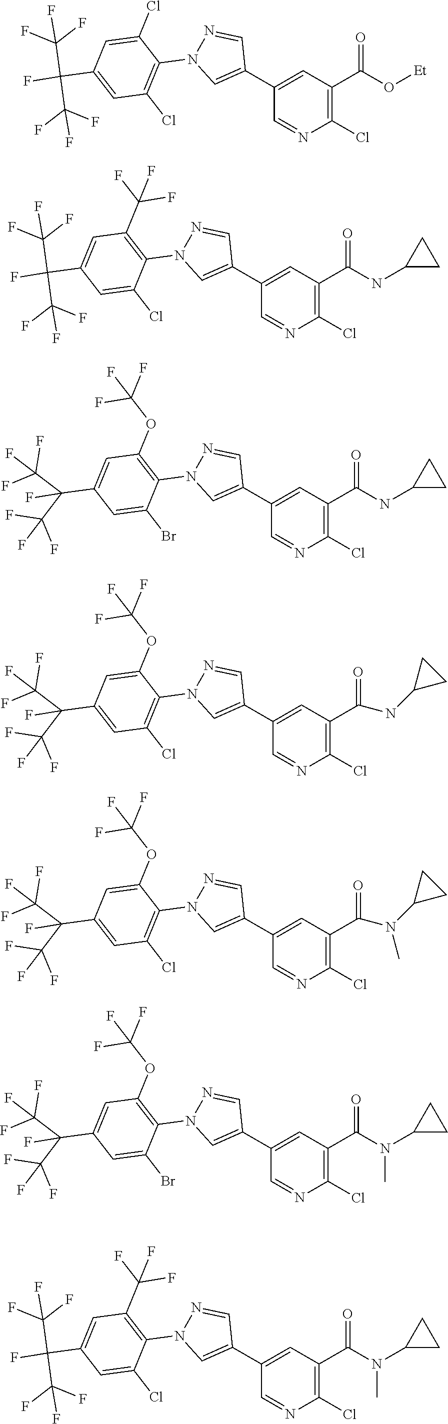

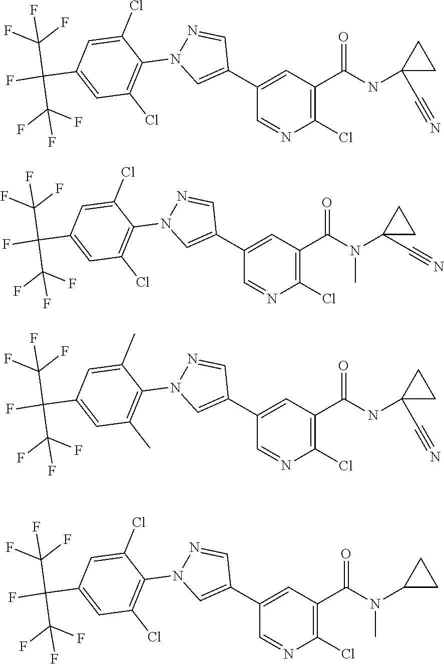

In the context of the present invention, unless defined differently elsewhere, the term “alkyl”, according to the invention, either on its own or else in combination with further terms, for example haloalkyl, is understood to mean a radical of a saturated, aliphatic hydrocarbon group which has 1 to 12, preferably 1 to 6 and particularly preferably 1 to 4 carbon atoms and may be branched or unbranched. Examples of C₁-C₁₂-alkyl radicals are methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1-ethylpropyl, 1,2-dimethylpropyl, hexyl, n-heptyl, n-octyl, n-nonyl, n-decyl, n-undecyl and n-dodecyl.

The term “alkoxy”, either on its own or else in combination with further terms, for example haloalkoxy, is understood in the present case to mean an O-alkyl radical, where the term “alkyl” is as defined above.

According to the invention, unless defined differently elsewhere, the term “aryl” is understood to mean an aromatic radical having 6 to 14 carbon atoms, preferably phenyl, naphthyl, anthryl or phenanthrenyl, particularly preferably phenyl.

According to the invention, unless defined differently elsewhere, the term “cycloalkyl”, either on its own or else in combination with further terms, is understood to mean a C₃-C₈-cycloalkyl radical, for example cyclopropyl, cyclobutyl, cyclopentyl, cyclohexyl, cycloheptyl and cyclooctyl.

Halogen-substituted radicals, for example haloalkyl, are mono- or polyhalogenated up to the maximum possible number of substituents. In the case of polyhalogenation, the halogen atoms may be identical or different. Unless stated otherwise, optionally substituted radicals may be mono- or polysubstituted, where the substituents in the case of polysubstitutions may be the same or different.

The ranges specified above generally or in preferred ranges apply correspondingly to the overall process. These definitions can be combined with one another as desired, i.e. including combinations between the respective preferred ranges.

Preference according to the invention is given to using processes in which there is a combination of the meanings and ranges specified above as being preferred.

Particular preference according to the invention is given to using processes in which there is a combination of the meanings and ranges specified above as being particularly preferred.

Very particular preference according to the invention is given to using processes in which there is a combination of the meanings and ranges specified above as being very particularly preferred.

Especially used according to the invention are processes in which there is a combination of the meanings and ranges specified above with the term “especially”.

Specifically used according to the invention are processes in which there is a combination of the meanings and ranges specified above with the term “specifically”.

Process Description

Step (1):

According to the invention, the compounds of the formula (II) are reacted in a first step with a magnesium- or lithium-based metallation reagent to give compounds of the formula (III),

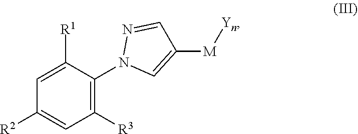

where R¹, R² and R³ are as defined above, Y is chlorine, bromine or iodine, preferably chlorine or bromine, n is 0 or 1 and M is magnesium (when n=1) or lithium (when n=0).

Preferably, in addition, M is magnesium and n is 1.

Suitable magnesium- or lithium-based metallation reagents are especially alkyllithium compounds LiR, where R is C₁-C₆-alkyl, and alkylmagnesium halide compounds RMgHal, where R is C₁-C₆-alkyl and Hal is halogen, preferably chlorine, bromine or iodine. Preference is given to using n-butyllithium, sec-butyllithium, tert-butyllithium, n-hexyllithium, methylmagnesium chloride, bromide or iodide, ethylmagnesium chloride, bromide or iodide or (n- or iso)propylmagnesium chloride, bromide or iodide, particular preference is given to using n-butyllithium, n-hexyllithium, methylmagnesium chloride or bromide, ethylmagnesium chloride or bromide or isopropylmagnesium chloride or bromide, and very particular preference is given to using methylmagnesium bromide or chloride, ethylmagnesium bromide or chloride or isopropylmagnesium chloride or bromide. It is also possible to use mixtures of the reagents mentioned.

The reactivity of the metallation reagent may optionally be increased by adding lithium chloride; the metallation of compounds of the general formula (II) to give compounds of the formula (III) is preferably conducted according to the invention without the addition of activating agents.

Preferably amounts of between 0.8 and 2.0 equivalents, particularly preferably between 0.9 and 1.5 equivalents, very particularly preferably between 1.0 and 1.2 equivalents, based on the total molar amount of the compounds of the formula (II) used, of the magnesium- or lithium-based metallation reagents are used here.

Preference is given to using the metallation reagent in its commercially available form, in the case of lithium reagents as a solution in a nonpolar solvent such as hydrocarbons (e.g. n-pentane, n-hexane, n-heptane, cyclohexane) or aromatic solvents (e.g. toluene or trifluoromethylbenzene) and in the case of magnesium reagents as a solution in ethereal solvents (e.g. diethyl ether, methyl tert-butyl ether, tetrahydrofuran (THF) or methyl-THF), without further dilution. The metallation reagent is preferably used at concentrations of from 0.2 mol/1 to 5.0 mol/l, particularly preferably at concentrations of from 0.2 mol/1 to 3.0 mol/1 and very particularly preferably at concentrations of from 0.5 mol/1 to 3.0 mol/l.

According to the invention, preference is given to metering the metallation reagent as a solution in the diluent or solvent defined above as preferred into a solution of the compound of the formula (II) in a diluent or solvent according to the invention as defined below for step (1). Inverse metering is also possible, but is less preferred for technical reasons.

The reaction time for the metallation is preferably in the region of the metering time for the metallation reagent. The reaction is instantaneous. Those skilled in the art can estimate the metering time without problems based on experience. However, the metering preferably takes place over from 0.5 to 6 hours, particularly preferably from 1 to 4 hours. Longer metering times are also possible from a technical point of view but are not expedient from an economic point of view.

The reaction can be carried out within a wide temperature range. Usually, it is conducted within a temperature range of −78 to 200° C., preferably at temperatures from −20 to 100° C., particularly preferably at temperatures from −10 to 50° C.

The reaction can be carried out at elevated or else reduced pressure. However, it is preferably conducted at standard pressure, e.g. in the range of 1013 hPa±300 hPa, or in the range of 1013 hPa±100 hPa, or in the range of 1013 hPa 50 hPa.

Step (1) is preferably conducted in a suitable diluent or solvent. Suitable solvents are in principle all organic solvents which are inert under the specific reaction conditions, such as for example aliphatic, cycloaliphatic or aromatic hydrocarbons (e.g. pentane, hexane, heptane, octane, nonane and technical grade hydrocarbons, cyclohexane, methylcyclohexane, petroleum ether, ligroin, benzene, toluene, trifluoromethylbenzene, xylene, mesitylene), aliphatic, cycloaliphatic or aromatic ethers (e.g. 1,2-dimethoxyethane (DME), diglyme, tetrahydrofuran (THF), 2-methyl-THF, 1,4-dioxane, methyl tert-butyl ether, anisole) or mixtures of the stated solvents.

Preferred solvents are hexane, heptane, cyclohexane, methylcyclohexane, benzene, toluene, trifluoromethylbenzene, xylene, mesitylene, anisole, THF, 2-methyl-THF or methyl tert-butyl ether, particular preference is given to toluene, trifluoromethylbenzene, xylene, anisole, THF or methyl tert-butyl ether.

Step (2):

In the process according to the invention, transmetallation of the compounds of the formula (III) with an inorganic zinc compound takes place after step (1) to give compounds of the formula (IV),

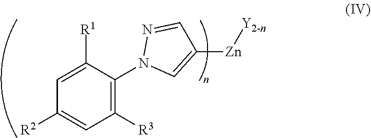

where R¹, R², R³ and Y are as defined above and n is 1 or 2.

This is particularly advantageous since the zinc reagents formed have improved stability and selectivity, and therefore the compounds of the general formula (I) can be obtained with higher yields and purities.

Inorganic zinc compounds that are suitable for the transmetallation are especially zinc halides and zinc acetate. Preference is given to using zinc chloride, zinc bromide, zinc iodide and zinc acetate, particular preference is given to using zinc chloride and zinc bromide, and very particular preference is given to using zinc chloride. It is also possible to use mixtures of the reagents mentioned.

Preferably amounts of between 0.4 and 2.0 equivalents, particularly preferably between 0.45 and 1.5 equivalents, very particularly preferably between 0.5 and 1.2 equivalents, based on the total molar amount of the compounds of the formula (III) used, of the inorganic zinc compound are used here.

The zinc-based transmetallation reagent is preferably used here in pure form or as a solution in a suitable ethereal solvent (e.g. THF, 2-methyl-THF or methyl tert-butyl ether) at concentrations of from 0.05 mol/l to 3.0 mol/l, particularly preferably in pure form or as an ethereal solution at concentrations of from 0.2 mol/1 to 2.5 mol/1 and very particularly preferably in pure form or as an ethereal solution at concentrations of from 0.5 mol/1 to 1.5 mol/l.

According to the invention, the transmetallation can preferably take place by adding a solution of the inorganic zinc salt to a solution of the compounds of the general formula (III) in one of the solvents stated above under step (1) or by inverse metering.

The duration of the metering can be in a preferred range from 0.1 to 4 hours, particularly preferably from 0.2 to 2 hours. Longer metering times are also possible from a technical point of view but are not expedient from an economic point of view.

The reaction can be carried out within a wide temperature range. Usually, it is conducted within a temperature range of −78 to 200° C., preferably at temperatures from −20 to 100° C., particularly preferably at temperatures from −10 to 50° C.

The reaction can be carried out at elevated or else reduced pressure. However, it is preferably conducted at standard pressure, e.g. in the range of 1013 hPa±300 hPa, or in the range of 1013 hPa±100 hPa, or in the range of 1013 hPa 50 hPa.

Step (2) is preferably conducted in a suitable diluent or solvent. Suitable solvents are in principle all organic solvents which are inert under the specific reaction conditions, such as for example aliphatic, cycloaliphatic or aromatic hydrocarbons (e.g. pentane, hexane, heptane, octane, nonane and technical grade hydrocarbons, cyclohexane, methylcyclohexane, petroleum ether, ligroin, benzene, toluene, trifluoromethylbenzene, xylene, mesitylene), aliphatic, cycloaliphatic or aromatic ethers (e.g. 1,2-dimethoxyethane (DME), diglyme, tetrahydrofuran (THF), 2-methyl-THF, 1,4-dioxane, methyl tert-butyl ether, anisole) or mixtures of the stated solvents.

Preference is given to conducting step (2) in the same diluent or solvent as step (1).

Step (3):

Preparation of Compounds of the Formula (I)

The process according to the invention further comprises reacting the compounds of the formula (IV) with compounds of the formula (V),

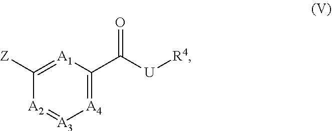

where A₁, A₂, A₃, A₄, R⁴, R⁵, U and Z are as defined above,

in the presence of at least one Pd(0) or Pd(II) compound and at least one monodentate or bidentate ligand, to give compounds of the formula (I).

Suitable Pd(0) or Pd(II) compounds are especially palladium(II) halides (preferably chloride, bromide, iodide), Pd(OAc)₂, palladium(II) pivalate, allylpalladium(II) chloride dimer, Pd(acac)₂ (acac=acetylacetonate), Pd(NO₃)₂, Pd(dba)₂ (dba=dibenzylideneacetone), Pd₂(dba)₃, dichlorobis(triphenylphosphine)palladium(II), Pd(dppf)Cl₂ (dppf=bis(diphenylphosphino)ferrocene), Pd(MeCN)₂Cl₂, and tetrakis(triphenylphosphine)palladium(0). Preference is given to PdCl₂, Pd(OAc)₂, allylpalladium(II) chloride dimer, Pd(acac)₂, Pd₂(dba)₃, dichlorobis(triphenylphosphine)palladium(II), Pd(dppf)Cl₂ and Pd(MeCN)₂Cl₂, particular preference is given to Pd(OAc)₂, palladium(II) pivalate, allylpalladium(II) chloride dimer, Pd₂(dba)₃, Pd(dppf)Cl₂ and Pd(MeCN)₂Cl₂ and very particular preference is given to using Pd(OAc)₂. It is also possible to use mixtures of the compounds mentioned.

Amounts of the Pd(0) or Pd(II) compound, based on the total molar amount of the compounds of the formula (IV) used, of between 0.0001 and 0.05 equivalent are preferably used here, particularly preferably between 0.0003 and 0.025 equivalent, very particularly preferably between 0.0004 and 0.01 equivalent.

Suitable monodentate or bidentate ligands are by way of example optionally mono- or polysubstituted triarylphosphines (especially triphenylphosphine (PPh₃), tris(o-tolyl)phosphine (P(o-tol)₃), tris(p-tolyl)phosphine (P(p-tol)₃), diarylalkylphosphines, dialkylarylphosphines (especially RuPhos (2-dicyclohexylphosphino-2′,6′-diisopropoxybiphenyl), CPhos (2-(2-dicyclohexylphosphanylphenyl)-N1,N1,N3,N3-tetramethylbenzyl-1,3-diamine), APhos (4-(N,N-dimethylamino)phenyl)di-tert-butylphosphine), DavePhos (2-dicyclohexylphosphino-2′-(N,N-dimethylamino)biphenyl), phenyl-DavePhos), trialkylphosphines (especially tris(tert-butyl)phosphine P(t-Bu)₃, tricyclohexylphosphine (P(Cy)₃)), diaryl(dialkylamino)phosphines, arylbis(dialkylamino)phosphines, diarylcycloalkylphosphines, BINAP (2,2′-bis(diphenylphosphino)-1,1′-binaphthalene), bis(diphenylphosphino)ferrocene (dppf), DPEPhos (oxydi-2,1-phenylene)bis(diphenylphosphine), XantPhos (4,5-bis(diphenylphosphino)-9,9-dimethylxanthene), tert-butyl-XantPhos or N-XantPhos. Preference is given to triphenylphosphine (PPh₃), tris(o-tolyl)phosphine (P(o-tol)₃), tris(cyclohexyl)phosphine (PCy₃), tris(tert-butyl)phosphine (P(t-Bu)₃), dppf, DPEPhos, XantPhos, tert-butyl-XantPhos or N-XantPhos, particularly preferably tris(cyclohexyl)phosphine (PCy₃), dppf, DPEPhos, XantPhos, tert-butyl-XantPhos or N-XantPhos and very particular preference is given to using XantPhos, tert-butyl-XantPhos or N-XantPhos. It is also possible to use mixtures of the compounds mentioned.

The molar ratio between metal and ligand can be varied widely, preference being given to using amounts of the ligands, based on the total molar amount of palladium used, of 1.0 to 6.0 equivalents, particularly preferably from 1.0 to 4.0 equivalents.

The palladium compounds and the ligands may be used according to the invention in pure form, each as a separate solution in a suitable diluent or solvent, as an isolated, preformed palladium/ligand complex, in pure form or as a solution in a suitable diluent or solvent, or as a joint mixture, with a molar ratio in accordance with the invention, in a suitable diluent or solvent. The palladium compounds and the ligands are preferably used each as a separate solution in a suitable diluent or solvent according to the invention, preferably at concentrations from 0.05 to 2.0% by weight, particularly preferably at concentrations between 0.1 and 1.5% by weight.

Suitable diluents or solvents are those defined below for step (3), preferably the same diluent or solvent is used as for step (3).

According to the invention, the compound of the general formula (V) is preferably used in amounts, based on the total molar amount of the compounds of the general formula (IV) used, of between 0.8 and 2.0 equivalents, particularly preferably between 0.85 and 1.5 equivalents, very particularly preferably between 0.9 and 1.2 equivalents.

The compound of the general formula (V) may be used as a solid or as a solution in an organic solvent according to the invention at concentrations of 5-40% by weight, preferably as a solid or as a solution in an organic solvent according to the invention at concentrations of 10-30% by weight, very particularly preferably as a solid or as a solution in an organic solvent according to the invention at concentrations of 15-30% by weight.

According to the invention, the coupling step (3) can preferably take place by adding the solution from step (2) to a solution of the compounds of the general formula (V) in one of the suitable solvents stated for step (3) or by inverse metering.

The reaction can be carried out within a wide temperature range. Usually, it is conducted within a temperature range of −78 to 200° C., preferably at temperatures from −10 to 150° C., particularly preferably from 15 to 120° C.

The reaction can be carried out at elevated or else reduced pressure. However, it is preferably conducted at standard pressure, e.g. in the range of 1013 hPa±300 hPa, or in the range of 1013 hPa±100 hPa, or in the range of 1013 hPa±50 hPa.

Step (3) is preferably conducted in a suitable diluent or solvent. Suitable solvents are in principle all organic solvents which are inert under the specific reaction conditions, such as for example aliphatic, cycloaliphatic or aromatic hydrocarbons (e.g. pentane, hexane, heptane, octane, nonane and technical grade hydrocarbons, cyclohexane, methylcyclohexane, petroleum ether, ligroin, benzene, toluene, trifluoromethylbenzene, xylene, mesitylene), aliphatic, cycloaliphatic or aromatic ethers (e.g. 1,2-dimethoxyethane (DME), diglyme, tetrahydrofuran (THF), 2-methyl-THF, 1,4-dioxane, methyl tert-butyl ether, anisole) or mixtures of the stated solvents.

Preference is given to conducting step (3) in the same diluent or solvent as step (1) and step (2).

The workup and isolation of the compounds (I) may, after complete reaction, take place for example by partially removing or without removing part of the solvent, washing with water or an aqueous acid and separating the organic phase, and removing the solvent under reduced pressure. The crude product may also possibly be recrystallized from a suitable solvent generally known to those skilled in that art or be precipitated by adding a further, second solvent generally known to those skilled in the art.

Compounds of the formula (I) where U=oxygen may be converted in an optional step (4), by commonly used methods known to those skilled in the art, into compounds of the formula (I) where U=N(R⁵) (where R⁵ is as defined above).

The amide formation may take place either directly by reacting with compounds of the formula H₂NR⁴ or take place via the preparation of compounds of the general formula (I) where U=oxygen and R⁴═H in the presence of at least one base. (See Scheme 1).

The amide formation may by way of example take place analogously to the methods described in WO2009/150230, WO2015/067646 or Org. Lett. 2011, 13, 5048.

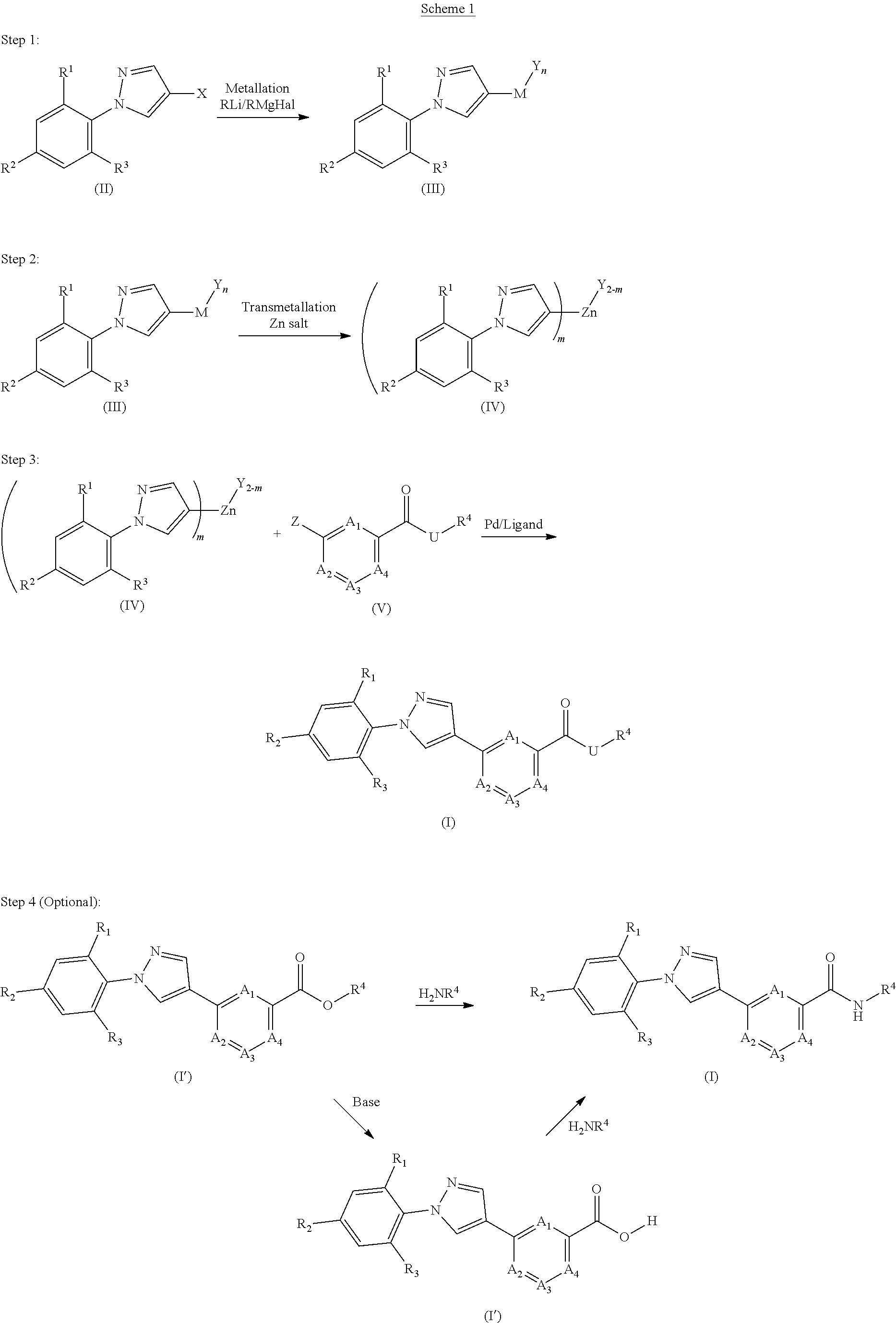

Scheme 1 gives a schematic overall representation of the process according to the invention including all steps. Reaction conditions and reactants are selected here in accordance with the above-described inventive and preferred configurations. All variables in the formulae (I), (I′), (II), (III), (IV) and (V) are defined as described above.

In one preferred configuration of the process according to the invention, the process comprises the steps (1), (2), (3) and optionally (4) or consists of these steps.

In one additionally preferred configuration of the process according to the invention, the steps (1), (2) and (3) take place in the same solvent or diluent.

In one particularly preferred configuration of the process according to the invention, no isolation and/or purification of the intermediates (III) and (IV) takes place between steps (1) and (2) and between (2) and (3).

If the process according to the invention comprises step (4), isolation and optionally purification of the compounds of the formula (I) where U=oxygen preferably takes place between step (3) and step (4).

Advantageously, during the whole process according to the invention, the solvent or diluent is never removed in its entirety, with the result that the intermediate compounds of the formulae (III) and (IV) are always in solution. Preferably less than 50% by volume (per cent by volume based on the volume of solvent used), particularly preferably less than 30% by volume, very particularly preferably less than 10% by volume and especially preferably at most 5% by volume of the solvent is removed (e.g. by evaporation, e.g. at a reaction temperature of about 40° C., or active removal, e.g. by distillation and/or reduced pressure based on 1013 hPa). Specifically, no solvent or diluent is removed during the whole process according to the invention.

One preferred embodiment of the process according to the invention is as follows:

The compounds of the general formula (II) are admixed, in a suitable organic solvent, with a metallation reagent according to the invention, e.g. ethylmagnesium bromide, at preferably −20° C. to 100° C., particularly preferably at −10° C. to 50° C. After addition is complete, an inorganic zinc compound, e.g. zinc chloride, for example dissolved in a suitable ethereal solvent, is metered into the reaction mixture at the same temperature, preferably over 0.1 to 4 hours, particularly preferably over 0.2 to 2 hours. (Step (1) and (2)). Preference is given to using the compounds of the formula (IV) directly in step (3) without further workup or isolation.

Preference is given to reacting the compounds of the formula (IV) subsequently in an organic solvent according to the invention, preferably in the same solvent as step (1) and (2), at preferably −10° C. to 150° C., particularly preferably at 15° C. to 120° C., with compounds of the formula (V) in the presence of a palladium source according to the invention, e.g. palladium acetate, and of a ligand according to the invention, e.g. Xantphos. (Step (3)) The compounds of the formula (I) formed can then be isolated and purified by the above-described methods.

One particularly preferred embodiment of the process according to the invention is as follows:

The compounds of the general formula (II) are initially charged in toluene or trifluoromethylbenzene and admixed with isopropylmagnesium chloride or bromide or ethylmagnesium chloride or bromide at −10° C. to 50° C. After addition is complete, zinc chloride, for example as a solution in tetrahydrofuran, is metered into the reaction mixture at the same temperature over 0.2 to 2 hours. (Step (1) and (2)). Preference is given to using the compounds of the formula (IV) directly in step (3) without further workup or isolation.

Preference is given to reacting the compounds of the formula (IV) subsequently in toluene or trifluoromethylbenzene at 15° C. to 120° C. with compounds of the formula (V) in the presence of palladium acetate and Xantphos. (Step (3)) The compounds of the formula (I) formed can then be isolated and purified by the above-described methods.

The present invention additionally relates to the intermediate compounds of the formulae (III) and (IV).

The invention provides compounds of the formula (III)

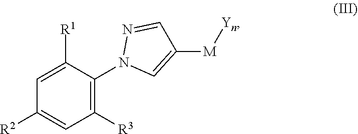

where R¹, R² and R³ are as defined above and where at most one of the radicals R¹ and R³ is methyl, Y is chlorine, bromine or iodine, preferably chlorine or bromine, n is 0 or 1 and M is magnesium (when n=1) or lithium (when n=0).

Preferably, in addition, M is magnesium and n is 1.

The invention further provides compounds of the formula (IV)

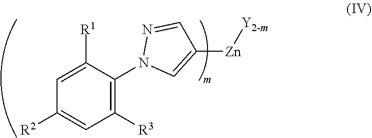

where R¹, R² and R³ are as defined above, Y is chlorine, bromine or iodine, preferably chlorine or bromine, and m is 1 or 2.

Also described herein are the compounds of the formula (I′), which do not form part of the subject-matter of the present invention,

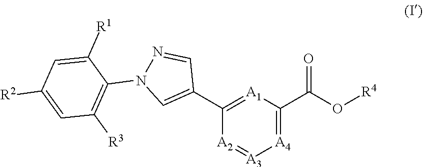

where

R¹ is halogen or C₁-C₃-alkyl,

R² is fluorine-substituted C₁-C₄-alkyl or fluorine-substituted C₁-C₄-alkoxy,

R³ is halogen, C₁-C₃-alkyl or fluorine-substituted C₁-C₃-alkyl, C₁-C₃-alkoxy or fluorine-substituted C₁-C₃-alkoxy,

A₁ is C—H,

A₂ is C—H,

A₃ is C—H or N and

A₄ is C—R₉, where

R₉ is halogen and

R⁴ is hydrogen, C₁-C₈-alkyl optionally substituted by halogen or CN, or C₃-C₆-cycloalkyl optionally substituted by halogen or CN, and where, if R¹ and R³ are simultaneously methyl, A₃ is N.

Preferably,

R¹ is Br or Cl,

R² is heptafluoroisopropyl,

R³ is Cl, methyl, trifluoromethyl, trifluoromethoxy or difluoromethoxy,

A₁ is C—H,

A₂ is C—H,

A₃ is N and

A₄ is C—Cl and

R⁴ is hydrogen or C₁-C₈-alkyl, especially hydrogen or C₁-C₆-alkyl.

Particularly preferred here are the compounds of the formulae (I′-1) to (I′-4),

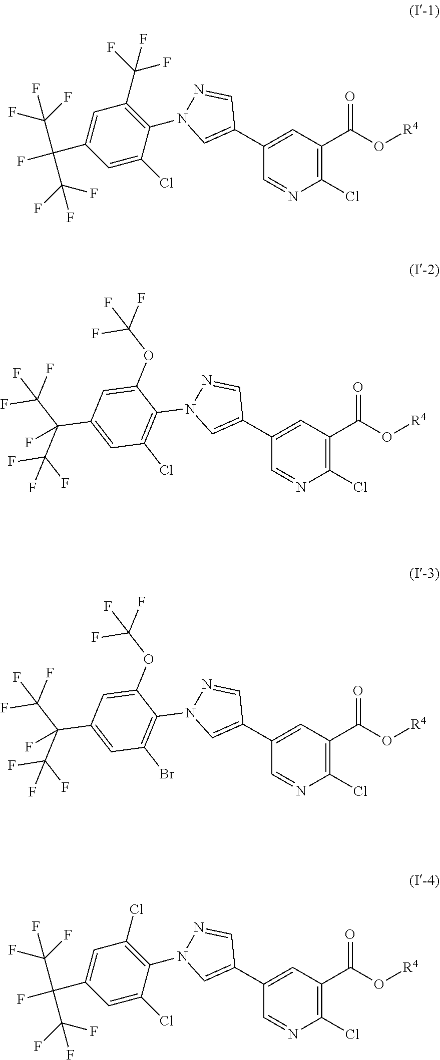

where

R⁴ is hydrogen, C₁-C₈-alkyl optionally substituted by halogen or CN, or C₃-C₆-cycloalkyl optionally substituted by halogen or CN, preferably hydrogen or C₁-C₆-alkyl and particularly preferably methyl or ethyl.

### EXAMPLES

The following examples explain the process according to the invention in more detail without limiting the invention thereto.

Methods:

The NMR data of the examples are listed in conventional form (6 values, multiplet splitting, number of hydrogen atoms).

The solvent and the frequency in which the NMR spectrum was recorded are stated in each case.

^{a)} HPLC (High Performance Liquid Chromatography) on a reversed-phase column (C18), Agilent 1100 LC system; Phenomenex Prodigy 100×4 mm ODS3; eluent A: acetonitrile (0.25 ml/l); eluent B: water (0.25 ml TFA/1); linear gradient from 5% acetonitrile to 95% acetonitrile in 7.00 min, then 95% acetonitrile for a further 1.00 min; oven temperature 40° C.; flow rate: 2.0 ml/min.

### Preparation of the Compounds of the General Formula (I)

### Example 1) 2-chloro-N-cyclopropyl-5-[1-[2,6-dichloro-4-[1,2,2,2-tetrafluoro-1-(trifluoromethyl)-ethyl]phenyl]pyrazol-4-yl]-N-methylpyridine-3-carboxamide (I-1)

50.0 g (97.6 mmol, 1.0 eq) of 1-[2,6-dichloro-4-[1,2,2,2-tetrafluoro-1-(trifluoromethyl)ethyl]phenyl]-4-iodo-1H-pyrazole were initially charged in 200 ml of toluene and admixed with 102.5 ml (1 M in THF, 102.5 mmol, 1.05 eq) of ethylmagnesium bromide over 45 min at 0-5° C. Subsequently, 72.5 ml (0.7 M in THF, 50.7 mmol, 0.5 eq) of zinc chloride, diluted with a further 150 ml of tetrahydrofuran, were metered in over 75 min at this temperature. After metering was complete, the reaction mixture was warmed to room temperature and stirred at this temperature for 10 min. The organozinc reagent thus prepared was subsequently metered into a solution of 28.8 g (97.6 mmol, 1.0 eq) of 5-bromo-2-chloro-N-cyclopropyl-N-methylpyridine-3-carboxamide, 11.0 mg (0.05 mmol, 0.0005 eq) of Pd(OAc)₂ and 56.5 mg (0.1 mmol, 0.001 eq) of XantPhos in 50 ml of toluene and 50 ml of tetrahydrofuran over 80 min at 70° C. The reaction mixture was stirred for 2 h at this temperature and, after adding 250 ml of toluene, tetrahydrofuran was removed by distillation. The organic phase was washed with 400 ml of 10% by weight HCl and the aqueous phase was extracted once with 150 ml of toluene. The combined organic phases were treated once with 16 g of N-acetylcysteine, dissolved in 500 ml of water, and subsequently washed with 500 ml of water. After removing the solvent under reduced pressure, suspending the residue in 200 ml of n-heptane for 1 h at 60° C., cooling to room temperature and filtering and drying under reduced pressure at 40° C., the product was obtained as a beige solid: yield 46.0 g (79% of theory).

¹H-NMR (CDCl₃, 400 MHz) δ (ppm)=8.63 (d, J=2.5 Hz, 1H), 8.13 (br s, 1H), 7.91 (d, J=2.5 Hz, 1H), 7.80 (br s, 1H), 7.75 (s, 2H), 3.16 (s, 3H), 2.80-2.95 (m, 1H), 0.55-1.00 (m, 4H).

### Example 2) 2-chloro-N-cyclopropyl-5-[1-[2,6-dichloro-4-[1,2,2,2-tetrafluoro-1-(trifluoromethyl)-ethyl]phenyl]pyrazol-4-yl]-N-methylpyridine-3-carboxamide (I-1)

2.5 g (4.73 mmol, 1.0 eq) of 1-[2,6-dichloro-4-[1,2,2,2-tetrafluoro-1-(trifluoromethyl)ethyl]phenyl]-4-iodo-1H-pyrazole were initially charged in 10 ml of trifluorotoluene and admixed with 4.97 ml (1 M in THF, 4.97 mmol, 1.05 eq) of ethylmagnesium bromide over 15 min at 0-5° C. Subsequently, 3.55 ml (0.7 M in THF, 2.48 mmol, 0.53 eq) of zinc chloride were metered in over 15 min at this temperature. After metering was complete, the reaction mixture was warmed to room temperature and stirred at this temperature for 10 min. The organozinc reagent thus prepared was subsequently metered into a solution of 1.6 g (4.7 mmol, 1.0 eq) of 5-bromo-2-chloro-N-cyclopropyl-N-methylpyridine-3-carboxamide, 0.4 mg (1.89 μmol, 0.0004 eq) of Pd(OAc)₂ and 2.2 mg (3.78 μmol, 0.0008 eq) of XantPhos in 2.5 ml of trifluorotoluene and 2.5 ml of tetrahydrofuran over 30 min at 70° C. The reaction mixture was stirred for 2 h at this temperature and, after adding 50 ml of toluene, tetrahydrofuran was removed by distillation. The organic phase was washed with 100 ml of 10% by weight HCl and the aqueous phase was extracted once with 100 ml of toluene. The combined organic phases were washed twice with 100 ml of water. After removing the solvent under reduced pressure, suspending the residue in 20 ml of n-heptane for 1 h at RT and filtering and drying under reduced pressure at 40° C., the product was obtained as a beige solid: yield 2.1 g (72% of theory).

¹H-NMR (CDCl₃, 400 MHz) δ (ppm)=8.63 (d, J=2.5 Hz, 1H), 8.13 (br s, 1H), 7.91 (d, J=2.5 Hz, 1H), 7.80 (br s, 1H), 7.75 (s, 2H), 3.16 (s, 3H), 2.80-2.95 (m, 1H), 0.55-1.00 (m, 4H).

### Example 3) 2-chloro-N-cyclopropyl-5-[1-[2,6-dichloro-4-[1,2,2,2-tetrafluoro-1-(trifluoromethyl)-ethyl]phenyl]pyrazol-4-yl]pyridine-3-carboxamide (I-2)

0.2 g (0.37 mmol, 1.0 eq) of 2-chloro-5-[1-[2,6-dichloro-4-[1,2,2,2-tetrafluoro-1-(trifluoromethyl)ethyl]phenyl]pyrazol-4-yl]pyridine-3-carboxylic acid and 27 mg (0.37 mmol, 1.0 eq) of N,N-dimethylformamide were initially charged in 5.0 ml of toluene and admixed with 133 mg (1.11 mmol, 3.0 eq) of thionyl chloride at 50° C. After 1 h at 50° C., the volatile constituents were removed under reduced pressure and the residue was taken up in 5.0 ml of acetonitrile, and 23 mg (0.41 mmol, 1.1 eq) of cyclopropylamine were added. 42 mg (0.41 mmol, 1.1 eq) of triethylamine were added at 0° C. and the reaction was warmed to 21° C. over 1 h. Subsequently, 45 mg (0.55 mmol, 1.5 eq) of 50% by weight NaOH were slowly added dropwise, the volatile constituents were removed under reduced pressure and the residue was stirred with 20 ml of dichloromethane. The organic phase was separated off, the solvent was removed under reduced pressure and the product was obtained as a yellow oil: yield: 160 mg (75% of theory).

¹H-NMR (CDCl₃, 400 MHz) δ (ppm)=8.65 (d, J=2.5 Hz, 1H), 8.30 (d, J=2.5 Hz, 1H), 8.16 (d, 0.7 Hz, 1H), 7.95 (d, J=0.7 Hz, 1H), 7.75 (s, 2H), 6.68 (br s, 1H), 2.95-2.99 (m, 1H), 0.93-0.95 (m, 2H), 0.69-0.71 (m, 2H).

### Example 4) 2-chloro-N-(1-cyanocyclopropyl)-5-[1-[2,6-dichloro-4-[1,2,2,2-tetrafluoro-1-(trifluoromethyl)ethyl]phenyl]pyrazol-4-yl]pyridine-3-carboxamide (I-3)

0.2 g (0.37 mmol, 1.0 eq) of 2-chloro-5-[1-[2,6-dichloro-4-[1,2,2,2-tetrafluoro-1-(trifluoromethyl)ethyl]phenyl]pyrazol-4-yl]pyridine-3-carboxylic acid and 27 mg (0.37 mmol, 1.0 eq) of N,N-dimethylformamide were initially charged in 5.0 ml of toluene and admixed with 133 mg (1.11 mmol, 3.0 eq) of thionyl chloride at 50° C. After 1 h at 50° C., the volatile constituents were removed under reduced pressure and the residue was taken up in 5.0 ml of acetonitrile, and 53 mg (0.44 mmol, 1.2 eq) of 1-aminocyclopropanecarbonitrile hydrochloride were added. 94 mg (0.93 mmol, 2.5 eq) of triethylamine were added at 0° C. and the reaction was warmed to 21° C. over 1 h. Subsequently, 45 mg (0.55 mmol, 1.5 eq) of 50% by weight NaOH were slowly added dropwise, the volatile constituents were removed under reduced pressure and the residue was stirred with 20 ml of dichloromethane. The organic phase was separated off, the solvent was removed under reduced pressure and the product was obtained as a yellow oil: yield: 160 mg (72% of theory).

¹H-NMR (CDCl₃, 400 MHz) δ (ppm)=8.70 (d, J=2.5 Hz, 1H), 8.39 (d, J=2.5 Hz, 1H), 8.18 (d, J=0.8 Hz, 1H), 7.97 (d, J=0.8 Hz, 1H), 7.75 (s, 2H), 1.71-1.74 (m, 2H), 1.42-1.46 (m, 2H).

### Example 5) methyl 2-chloro-5-[1-[2,6-dichloro-4-[1,2,2,2-tetrafluoro-1-(trifluoromethyl)ethyl]-phenyl]pyrazol-4-yl]pyridine-3-carboxylate (I-4)

9.0 g (17.6 mmol, 1.0 eq) of 1-[2,6-dichloro-4-[1,2,2,2-tetrafluoro-1-(trifluoromethyl)ethyl]phenyl]-4-iodo-1H-pyrazole were initially charged in 40 ml of toluene and admixed with 19.3 ml (1 M in THF, 19.3 mmol, 1.05 eq) of ethylmagnesium bromide over 40 min at 0-5° C. Subsequently, 13.6 ml (0.7 M in THF, 9.1 mmol, 0.5 eq) of zinc chloride, diluted with a further 20 ml of tetrahydrofuran, were metered in over 25 min at this temperature. After metering was complete, the reaction mixture was warmed to room temperature. The organozinc reagent thus prepared was subsequently metered into a solution of 4.6 g (17.6 mmol, 1.0 eq) of methyl 5-bromo-2-chloropyridine-3-carboxylate, 39.5 mg (0.2 mmol, 0.01 eq) of Pd(OAc)₂ and 203 mg (0.4 mmol, 0.02 eq) of XantPhos in 10 ml of toluene and 10 ml of tetrahydrofuran over 30 min at 70° C. The mixture was stirred at this temperature for 4 h. After adding 50 ml of toluene, the organic phase was washed with 100 ml of 10% by weight HCl and twice with 100 ml each time of water. After drying over sodium sulfate and removing the solvent, the residue was treated with 80 ml of n-heptane and, after filtering and drying under reduced pressure at 40° C., the product was obtained as a beige solid: yield 6.5 g (65% of theory).

¹H-NMR (CDCl₃, 400 MHz) δ (ppm)=8.72 (d, J=2.5 Hz, 1H), 8.31 (br s, 1H), 8.17 (d, J=2.5 Hz, 1H), 7.96 (br s, 1H), 7.75 (s, 1H), 4.00 (s, 3H).

### Example 6) 2-chloro-5-[1-[2,6-dichloro-4-[1,2,2,2-tetrafluoro-1-(trifluoromethyl)ethyl]phenyl]-pyrazol-4-yl]pyridine-3-carboxylic acid (I-5)

1.1 g (1.8 mmol, 1.0 eq) of methyl 2-chloro-5-[1-[2,6-dichloro-4-[1,2,2,2-tetrafluoro-1-(trifluoromethyl)ethyl]phenyl]pyrazol-4-yl]pyridine-3-carboxylate were dissolved in a mixture of 10 ml of THF and 10 ml of methanol and admixed with 0.07 g (2.75 mmol, 1.5 eq) of LiOH at 21° C. The mixture was stirred for 12 h at room temperature and, after addition of 50 ml of 20% by weight HCl, the product was obtained after filtration as a colourless solid: yield 0.9 g (90% of theory).

¹H-NMR (CDCl₃, 400 MHz) δ (ppm)=8.78 (d, J=2.5 Hz, 1H), 8.47 (d, J=2.5 Hz, 1H), 8.19 (d, J=0.7 Hz, 1H), 7.99 (d, J=0.7 Hz, 1H), 7.75 (s, 2H).

### Comparative Examples of Suzuki Coupling Analogously to WO2016/174052

### 2-Chloro-N-cyclopropyl-5-[1-[2,6-dichloro-4-[1,2,2,2-tetrafluoro-1-(trifluoromethyl)ethyl]-phenyl]pyrazol-4-yl]pyridine-3-carboxamide (I-2)

4.0 g (7.7 mmol, 1.0 eq) of 1-[2,6-dichloro-4-[1,2,2,2-tetrafluoro-1-(trifluoromethyl)ethyl]phenyl]-4-iodo-1H-pyrazole, 2.23 g (9.27 mmol, 1.2 eq) of 6-chloro-5-(cyclopropylcarbamoyl)-3-pyridyl]boronic acid were initially charged under argon in 35 ml of ethanol and 90 ml of toluene and admixed with a solution of 4.0 g (28.9 mmol, 3.75 eq) of potassium carbonate in 25 ml of degassed water. The reaction mixture was stirred for 1 h at RT, admixed with 379 mg (0.46 mmol, 0.06 eq) of PdCl₂(dppf)*CH₂Cl₂ and subsequently stirred at 50° C. After 3 h at this temperature, complete conversion to the desired product was detected by means of HPLC^{a)}. After cooling to RT, the phases were separated, the aqueous phase was extracted with toluene, and the combined organic phases were washed once with a solution of 3.8 g of N-acetylcysteine in 100 ml of water, then with 100 ml of 10% by weight NaOH and finally with 100 ml of saturated NaCl solution. After drying over sodium sulfate and distillation of the solvent under reduced pressure, the product was obtainable as a beige-brown solid: yield 3.77 g (75% of theory).

### 2-Chloro-N-cyclopropyl-5-[1-[2,6-dichloro-4-[1,2,2,2-tetrafluoro-1-(trifluoromethyl)ethyl]-phenyl]pyrazol-4-yl]pyridine-3-carboxamide (I-2)

4.0 g (7.7 mmol, 1.0 eq) of 1-[2,6-dichloro-4-[1,2,2,2-tetrafluoro-1-(trifluoromethyl)ethyl]phenyl]-4-iodo-1H-pyrazole, 2.23 g (9.27 mmol, 1.2 eq) of 6-chloro-5-(cyclopropylcarbamoyl)-3-pyridyl]boronic acid were initially charged under argon in 35 ml of ethanol and 90 ml of toluene and admixed with a solution of 4.0 g (28.9 mmol, 3.75 eq) of potassium carbonate in 25 ml of degassed water. The reaction mixture was stirred for 1 h at RT, admixed with 63 mg (0.7 mmol, 0.01 eq) of PdCl₂(dppf)*CH₂Cl₂ and subsequently stirred at 50° C. After 10 h at this temperature, only incomplete conversion of 56% to the desired product was detectable by means of HPLC^{a}). The product was not isolated.

The following tricyclic compounds of the general formula (I) were preparable analogously to examples (1), (3), (5) and (6):

### Ethyl 2-chloro-5-[1-[2,6-dichloro-4-[1,2,2,2-tetrafluoro-1-(trifluoromethyl)ethyl]phenyl]pyrazol-4-yl]pyridine-3-carboxylate (I-6)

¹H-NMR (CDCl₃, 400 MHz) δ (ppm)=8.71 (d, J=2.5 Hz, 1H), 8.29 (d, J=2.5 Hz, 1H), 8.17 (s, 1H), 7.96 (s, 1H), 7.52 (s, 2H), 4.48 (q, J=7.2 Hz, 1H), 1.43 (t, J=7.2 Hz, 1H).

### 2-Chloro-5-[1-[2-chloro-4-[1,2,2,2-tetrafluoro-1-(trifluoromethyl)ethyl]-6-(trifluoromethoxy)phenyl]pyrazol-4-yl]-N-cyclopropyl-N-methylpyridine-3-carboxamide (I-7)

¹H-NMR (CDCl₃, 400 MHz) δ (ppm)=8.62 (d, J=2.5 Hz, 1H), 8.12 (s, 1H), 7.92 (s, 1H), 7.80 (s, 1H), 7.77 (d, J=2.5 Hz, 1H), 7.63 (s, 1H), 3.16 (s, 3H), 2.82-2.86 (m, 1H), 1.24-12.6 (m, 2H), 0.62 (br s, 2H).

### 2-Chloro-5-[1-[2-chloro-4-[1,2,2,2-tetrafluoro-1-(trifluoromethyl)ethyl]-6-(trifluoromethyl)phenyl]pyrazol-4-yl]-N-cyclopropylpyridine-3-carboxamide (I-8)

¹H-NMR (CDCl₃, 400 MHz) δ (ppm)=8.65 (d, J=2.5 Hz, 1H), 8.29 (d, J=2.5 Hz, 1H), 8.15 (s, 1H), 8.04 (br s, 1H), 7.99 (br s, 1H), 7.94 (s, 1H), 6.68 (br s, 1H), 3.00-2.92 (m, 1H), 0.96-0.91 (m, 2H), 0.71-0.69 (m, 2H).

### 5-[1-[2-Bromo-4-[1,2,2,2-tetrafluoro-1-(trifluoromethyl)ethyl]-6-(trifluoromethoxy)phenyl]pyrazol-4-yl]-2-chloro-N-cyclopropyl-N-methylpyridine-3-carboxamide (I-9)

¹H-NMR (CDCl₃, 400 MHz) δ (ppm)=8.61 (d, J=2.4 Hz, 1H), 8.07 (s, 1H), 7.88 (s, 1H), 7.76 (d, J=2.4 Hz, 1H), 7.57 (br s, 1H), 7.53 (s, 1H), 6.68 (br s, 1H), 3.17 (s, 3H), 3.00-2.92 (m, 1H), 0.96-0.91 (m, 2H), 0.65-0.56 (m, 2H).

### Ethyl 2-chloro-5-[1-[2-chloro-4-[1,2,2,2-tetrafluoro-1-(trifluoromethyl)ethyl]-6-(trifluoromethyl)phenyl]pyrazol-4-yl]pyridine-3-carboxylate (I-10)

¹H-NMR (CDCl₃, 400 MHz) δ (ppm)=8.70 (d, J=2.5 Hz, 1H), 8.27 (d, J=2.5 Hz, 1H), 8.16 (s, 1H), 8.04 (br s, 1H), 7.99 (br s, 1H), 7.96 (s, 1H), 4.47 (q, J=7.2 Hz, 2H), 1.45 (t, J=7.2 Hz, 3H).

### Ethyl 2-chloro-5-[1-[2-chloro-4-[1,2,2,2-tetrafluoro-1-(trifluoromethyl)ethyl]-6-(trifluoromethoxy)phenyl]pyrazol-4-yl]pyridine-3-carboxylate (I-11)

¹H-NMR (DMSO-d₆, 400 MHz) δ (ppm)=8.96 (d, J=2.5 Hz, 1H), 8.93 (s, 1H), 8.58 (s, 1H), 8.49 (d, J=2.5 Hz, 1H), 8.24 (s, 1H), 7.96 (s, 1H), 4.39 (q, J=7.1 Hz, 2H), 1.36 (t, J=7.1 Hz, 3H).

### Methyl 2-chloro-5-[1-[2-chloro-4-[1,2,2,2-tetrafluoro-1-(trifluoromethyl)ethyl]-6-(trifluoromethyl)phenyl]pyrazol-4-yl]pyridine-3-carboxylate (I-12)

¹H-NMR (DMSO-d₆, 400 MHz) δ (ppm)=8.95 (d, J=2.5 Hz, 2H), 8.89 (s, 1H), 8.57 (s, 1H), 8.52 (d, J=2.5 Hz, 2H), 3.92 (s, 3H).

### 2-Chloro-5-[1-[2-chloro-4-[1,2,2,2-tetrafluoro-1-(trifluoromethyl)ethyl]-6-(trifluoromethoxy)phenyl]pyrazol-4-yl]pyridine-3-carboxylic acid (I-13)

¹H-NMR (DMSO-d₆, 400 MHz) δ (ppm)=13.90 (br s, 1H), 8.92 (s, 1H), 8.91 (s, 1H), 8.58 (s, 1H), 8.48 (d, J=2.5 Hz, 1H), 8.24 (d, J=1.8 Hz, 1H), 7.96 (br s, 1H).

### Methyl 2-chloro-5-[1-[2-chloro-4-[1,2,2,2-tetrafluoro-1-(trifluoromethyl)ethyl]-6-(trifluoromethoxy)phenyl]pyrazol-4-yl]pyridine-3-carboxylate (I-14)

¹H-NMR (DMSO-d₆, 400 MHz) δ (ppm)=8.97 (d, J=2.4 Hz, 1H), 8.93 (s, 1H), 8.58 (s, 1H), 8.53 (d, J=2.4 Hz, 1H), 8.23 (brs, 1H), 7.96 (br s, 1H), 3.89 (s, 3H).

### Ethyl 5-[1-[2-bromo-4-[1,2,2,2-tetrafluoro-1-(trifluoromethyl)ethyl]-6-(trifluoromethoxy)phenyl]pyrazol-4-yl]-2-chloropyridine-3-carboxylate (I-15)

¹H-NMR (CDCl₃, 400 MHz) δ (ppm)=8.70 (d, J=2.5 Hz, 1H), 8.35 (d, J=2.5 Hz, 1H), 8.13 (s, 1H), 7.96 (s, 1H), 7.57 (br s, 1H), 7.53 (br s, 1H), 4.46 (q, J=7.1 Hz, 2H), 1.42 (t, J=7.1 Hz, 3H).

### 2-Chloro-5-[1-[2-chloro-4-[1,2,2,2-tetrafluoro-1-(trifluoromethyl)ethyl]-6-(trifluoromethyl)phenyl]pyrazol-4-yl]pyridine-3-carboxylic acid (I-16)

¹H-NMR (DMSO-d₆, 400 MHz) δ (ppm)=13.89 (br s, 1H), 8.93 (d, J=2.5 Hz, 1H), 8.90 (s, 1H), 8.59 (s, 1H), 7.51 (br s, 1H), 8.49 (d, J=2.5 Hz, 1H), 8.10 (br s, 1H).

### Methyl 5-[1-[2-bromo-4-[1,2,2,2-tetrafluoro-1-(trifluoromethyl)ethyl]-6-(trifluoromethoxy)phenyl]pyrazol-4-yl]-2-chloropyridine-3-carboxylate (I-17)

¹H-NMR (CDCl₃, 400 MHz) δ (ppm)=8.70 (d, J=2.5 Hz, 1H), 8.29 (d, J=2.5 Hz, 1H), 8.13 (s, 1H), 7.93 (s, 1H), 7.57 (s, 1H), 7.53 (s, 1H), 3.65 (s, 3H).

### 5-[1-[2-Bromo-4-[1,2,2,2-tetrafluoro-1-(trifluoromethyl)ethyl]-6-(trifluoromethoxy)phenyl]pyrazol-4-yl]-2-chloropyridine-3-carboxylic acid (I-18)

¹H-NMR (DMSO-d₆, 400 MHz) δ (ppm)=8.91 (d, J=2.4 Hz, 1H), 8.85 (s, 1H), 8.53 (s, 1H), 8.48 (d, J=2.4 Hz, 1H), 7.93 (s, 1H), 7.72 (s, 1H).

### 5-[1-[2-Bromo-4-[1,2,2,2-tetrafluoro-1-(trifluoromethyl)ethyl]-6-(trifluoromethoxy)phenyl]pyrazol-4-yl]-2-chloro-N-cyclopropylpyridine-3-carboxamide (I-19)

¹H-NMR (CDCl₃, 400 MHz) δ (ppm)=8.64 (d, J=2.5 Hz, 1H), 8.27 (d, J=2.5 Hz, 1H), 8.12 (s, 1H), 7.92 (s, 1H), 7.57 (s, 1H), 7.53 (s, 1H), 6.69 (br s, 1H), 3.00-2.93 (m, 1H), 0.93-0.90 (m, 2H), 0.72-0.68 (m, 1H).

### 2-Chloro-5-[1-[2-chloro-4-[1,2,2,2-tetrafluoro-1-(trifluoromethyl)ethyl]-6-(trifluoromethoxy)phenyl]pyrazol-4-yl]-N-cyclopropylpyridine-3-carboxamide (I-20)

¹H-NMR (DMSO-d₆, 400 MHz) δ (ppm)=8.88 (s, 1H), 8.82 (d, J=2.4 Hz, 1H), 8.70 (d, J=4.2 Hz, 1H), 8.57 (s, 1H), 8.23 (s, 1H), 8.20 (d, J=2.4 Hz, 1H), 7.97 (br s, 1H), 2.90-280 (m, 1H) 0.78-0.70 (m, 2H), 0.58-0.53 (m, 2H).

1: A process for preparing a compound of formula (I) 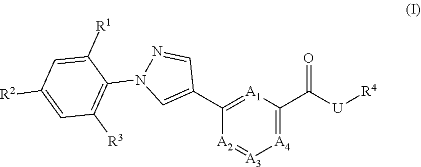
wherein
R¹ is hydrogen, cyano, halogen, C₁-C₄-alkyl optionally substituted by halogen or CN, or C₁-C₄-alkoxy optionally substituted by halogen,
R² is halogen, trifluoromethylsulfonyl, trifluoromethylsulfinyl, trifluoromethylsulfanyl, C₁-C₄-alkyl optionally substituted by halogen, or C₁-C₄-alkoxy optionally substituted by halogen, and
R³ is hydrogen, cyano, halogen, C₁-C₄-alkyl optionally substituted by halogen or CN, or C₁-C₄-alkoxy optionally substituted by halogen,
R⁴ is hydrogen, C₁-C₈-alkyl optionally substituted by halogen or CN, or C₃-C₆-cycloalkyl optionally substituted by halogen or CN,
U is oxygen or an N(R⁵) group, wherein
R⁵ is hydrogen, C₁-C₆-alkyl optionally substituted by halogen or CN, or C₃-C₆-cycloalkyl optionally substituted by halogen or CN,
A₁ is C—R₆,
A₂ is C—R₇,
A₃ is C—R₈ or N,
A₄ is C—R₉,
R₆, R₇, R₈ and R₉ are independently hydrogen, C₁-C₄-alkyl optionally substituted by halogen or CN, or halogen,
starting from compound of formula (II) 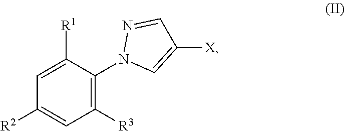
wherein R¹, R² and R³ are as defined above and X is iodine or bromine,
comprising the steps of
(1): reacting the compound of formula (II) with a magnesium- or lithium-based metallation reagent to give a compound of formula (III) 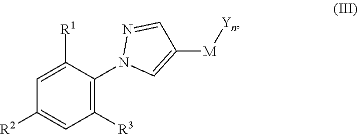
wherein R¹, R², R³ are as defined above, Y is chlorine, bromine or iodine, n is 0 or 1 and M is magnesium (when n=1) or lithium (when n=0),
(2): reacting the compound of formula (III) with an inorganic zinc compound to give a compound of formula (IV) 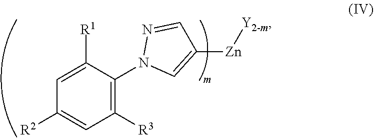
wherein R¹, R², R³ and Y are as defined above and m is 1 or 2, and
(3): reacting the compound of formula (IV) with a compound of formula (V) 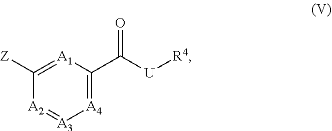
wherein A₁, A₂, A₃, A₄, R⁴, U and R⁵ are as defined above and Z is bromine or iodine,
in the presence of at least one Pd(0) or Pd(II) compound and at least one monodentate or bidentate ligand, to give the compound of formula (I).

2: The process according to claim 1, wherein
A₁ is C—H,
A₂ is C—H,
A₃ is C—H or N, and
A₄ is C—R₉,
and R₉ is C₁-C₄-alkyl optionally substituted by halogen or CN, or halogen.

3: The process according to claim 1, wherein A₃ is N.

4: The process according to claim 1, wherein at most one of the radicals R⁴ or R⁵ is hydrogen.

5: The process according to claim 1, wherein R² is halogen-substituted C₁-C₄-alkyl or halogen-substituted C₁-C₄-alkoxy.

6: The process according to claim 1, wherein R¹ and R³ in each case independently of one another are a substituent selected from hydrogen, Cl, Br, F, C₁-C₃-alkyl, halogen-substituted C₁-C₃-alkyl, C₁-C₃-alkoxy or halogen-substituted C₁-C₃-alkoxy.

7: The process according to claim 1, wherein R¹ and R³ are not simultaneously hydrogen in any compound.

8: The process according to claim 1, wherein
R¹ is halogen or C₁-C₃-alkyl,
R² is fluorine-substituted C₁-C₄-alkyl or fluorine-substituted C₁-C₄-alkoxy and
R³ is halogen, C₁-C₃-alkyl or fluorine-substituted C₁-C₃-alkyl, C₁-C₃-alkoxy or fluorine-substituted C₁-C₃-alkoxy.

9: The process according to claim 1, wherein the magnesium- or lithium-based metallation reagent in step (1) is selected from alkyllithium compounds LiR, wherein R is C₁-C₆-alkyl, and alkylmagnesium halide compounds RMgHal, wherein R is C₁-C₆-alkyl and Hal is halogen.

10: The process according to claim 1, wherein the amount of the Pd(0) or Pd(II) compound in step (3), based on the total molar amount of the compounds of the formula (IV) used, is between 0.0001 and 0.05 equivalent.

11: The process according to claim 1, wherein the inorganic zinc compound in step (2) is selected from zinc halides and zinc acetate.

12: The process according to claim 1, wherein the Pd(0) or Pd(II) compound in step (3) is selected from palladium(II) halides, Pd(OAc)₂, palladium(II) pivalate, allylpalladium(II) chloride dimer, Pd(acac)₂ (acac=acetylacetonate), Pd(NO₃)₂, Pd(dba)₂ (dba=dibenzylideneacetone), Pd₂(dba)₃, dichlorobis(triphenylphosphine)palladium(II), Pd(dppf)Cl₂ (dppf=bis(diphenylphosphino)ferrocene), Pd(MeCN)₂Cl₂, and tetrakis(triphenylphosphine)palladium(0).

13: The process according to claim 1, wherein the monodentate or bidentate ligands in step (3) are selected from optionally mono- or polysubstituted triarylphosphines, diarylalkylphosphines, dialkylarylphosphines, trialkylphosphines, diaryl(dialkylamino)phosphines, arylbis(dialkylamino)phosphines, diarylcycloalkylphosphines, BINAP (2,2′-bis(diphenylphosphino)-1,1′-binaphthalene), bis(diphenylphosphino)ferrocene (dppf), DPEPhos (oxydi-2,1-phenylene)bis(diphenylphosphine), XantPhos (4,5-bis(diphenylphosphino)-9,9-dimethylxanthene), tert-butyl-XantPhos or N-XantPhos.

14. The process according to claim 13, wherein the monodentate or bidentate ligands in step (3) are selected from triphenylphosphine (PPh₃), tris(o-tolyl)phosphine (P(o-tol)₃), tris(cyclohexyl)phosphine (PCy₃), tris(tert-butyl)phosphine (P(t-Bu)₃), dppf, DPEPhos, XantPhos, tert-butyl-XantPhos or N-XantPhos.

15: A compound of formula (III) 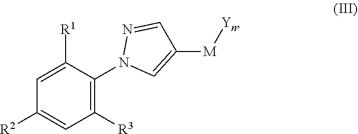
wherein R¹, R² and R³ are defined according to claim 1,
wherein at most one of the radicals R¹ and R³ is methyl, Y is chlorine, bromine or iodine, n is 0 or 1 and M is magnesium (when n=1) or lithium (when n=0).

16: A compound of formula (IV) 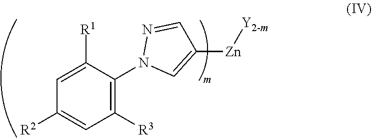
wherein R¹, R² and R³ are defined according to claim 1, Y is chlorine, bromine or iodine and m is 1 or 2.
